(19) United States
(12) Patent Application Publication
(10) Pub. No.: US 2026/0235606 A1
(43) Pub. Date: Aug. 13, 2026

(54) IMMUNOHISTOCHEMISTRY (IHC) PTK7 SCORING PROTOCOLS AND METHODS FOR AIDING CANCER TREATMENTS

(71) Applicant: AGILENT TECHNOLOGIES, INC., Santa Clara, CA (US)

(72) Inventors: Leah MACY, Santa Clara, CA (US); Jim CHRISTIAN, Santa Clara, CA (US); Edward MANNA, Santa Clara, CA (US); Donna KELL, Santa Clara, CA (US)

(73) Assignee: AGILENT TECHNOLOGIES, INC., Santa Clara, CA (US)

(21) Appl. No.: 19/155,834

(22) PCT Filed: Mar. 21, 2024
(86) PCT No.: PCT/US2024/020970

§ 371 Date: Aug. 12, 2025

Related U.S. Application Data
(60) Provisional application No. 63/453,890, filed on Mar. 22, 2023.

(51) Int. Cl. G01N 33/5752

(52) U.S. Cl. CPC G01N 33/5752; G01N 2333/912; G01N 2474/20

(57) ABSTRACT

In alternative embodiments, provided are immunohistochemistry (IHC) methods and kits for determining and scoring reproducibly the extent of expression of the protein tyrosine-protein kinase-like 7 (PTK7), also known as: colon carcinoma kinase 4 (CCK4); HER2 or receptor tyrosine-protein kinase erbB-2, or cluster of differentiation 340 (CD340); programmed death-ligand 1 (PD-L1), or cluster of differentiation 274 (CD274); B7 homolog 1 (B7-H1); and, Ki-67 or MKI67 (Marker of Proliferation Ki-67), in a tissue sample. In alternative embodiments, provided are methods and kits for diagnosing or selecting an individual eligible for treatment with a cancer or tumor therapeutic, or assessing the risk of recurrence for a cancer or a tumor using an IHC method as provided herein. In alternative embodiments, provided are kits comprising components and instructions for practicing methods as provided herein. The present application describes methods for scoring PTK7 expression and utilizing the score as a companion or complementary diagnostic, or to aid the treatment or amelioration of a cancer or a tumor.

### RELATED APPLICATIONS

This U.S. utility patent application claims the benefit of priority under 35 U.S.C. § 119(e) to U.S. Provisional Patent Application Serial No. (USSN) 63/453,890, Mar. 22, 2023. The aforementioned application is expressly incorporated herein by reference in their entirety and for all purposes.

### TECHNICAL FIELD

This invention generally relates to cancer treatments, companion or complementary diagnostics and immunohistochemical methods. In alternative embodiments, provided are immunohistochemistry (IHC) methods and kits for determining and scoring reproducibly the extent of expression of the protein tyrosine-protein kinase-like 7 (PTK7), also known as: colon carcinoma kinase 4 (CCK4); HER2 or receptor tyrosine-protein kinase erbB-2, or cluster of differentiation 340 (CD340); programmed death-ligand 1 (PD-L1), or cluster of differentiation 274 (CD274): B7 homolog 1 (B7-H1); and, Ki-67 or MK167 (Marker of Proliferation Ki-67), in a tissue sample. In alternative embodiments. provided are methods and kits for diagnosing or selecting an individual eligible for treatment with a cancer or tumor therapeutic or assessing the risk of recurrence for a cancer or a tumor using an IHC method as provided herein. In alternative embodiments, provided are kits comprising components and instructions for practicing methods as provided herein. The present application describes methods for scoring PTK7 expression and utilizing the score as a companion or complementary diagnostic or to aid the treatment or amelioration of a cancer or a tumor.

### BACKGROUND

Tyrosine-protein kinase-like 7 (PTK7), also known as colon carcinoma kinase 4 (CCK4), is a receptor tyrosine kinase that in humans is encoded by the PTK7 gene. PTK7 is expressed in and has been found to contribute to the progression of various cancers and tumors; for example, in lung cancers and non-small cell lung cancers (NSCLC). PTK7 overexpression has been found to be a candidate biomarker to predict the occurrence and prognosis of several types of cancers, including stage I-IV hepatocellular carcinoma (HCC), invasive breast cancers, cervical cancers, colorectal cancers, and thyroid cancers.

Manual scoring of qualitative immunohistochemistry (IHC) assays are described in pathology scoring manuals for companion diagnostic (CDx) assays. These scoring algorithms rely on subjective assessments of average IHC intensity and total percent positive tumor staining. These approaches cannot be accurately applied to intensity-based cutoffs that require a score of percent positive tumor at specific intensities on a scale from 0-3. There remains a need in the art for more objective and accurate approaches for scoring methods to assess PTK7 expression in tissue samples.

### SUMMARY

In alternative embodiments, provided are immunohistochemistry (IHC) methods for determining and scoring the extent of expression of protein tyrosine-protein kinase-like 7 (PTK7) (also known as: colon carcinoma kinase 4 (CCK4); HER2 or receptor tyrosine-protein kinase erbB-2, or cluster of differentiation 340 (CD340); programmed death-ligand 1 (PD-L1), or cluster of differentiation 274 (CD274); B7 homolog 1 (B7-H1); and, Ki-67 or MK167 (Marker of Proliferation Ki-67)) in a tissue sample, the method comprising:
(a) staining a tissue sample with an antibody which specifically binds to PTK7; and
(b) determining a total number of viable invasive tumor or cancer cells having anti-PTK7 staining, and determining a total number of staining and non-staining viable invasive tumor or cancer cells in at least a portion of the tissue sample, wherein an invasive tumor or cancer cell is counted as positively stained with anti-PTK7 if the invasive tumor or cancer cell displays at an anti-PTK7 cellular staining at any intensity above a defined threshold, and
(c) determining a Tumor Proportion Score (TPS),
wherein the Tumor Proportion Score (TPS) is the number of PTK7 staining viable invasive tumor or cancer cells found in the tissue sample divided by the total number of staining and non-staining viable invasive tumor or cancer cells, multiplied by 100.

In alternative embodiments of methods are provided herein:
the defined threshold comprises:
   a 1+ or greater positive staining intensity evaluated at a low magnification;
   a 2+ or greater positive staining intensity evaluated at a medium magnification; or
   a 3+ positive staining intensity evaluated at a high magnification;
the low magnification is at least about 4× magnification: the medium magnification is at least about 10× magnification; and the high magnification is at least about 20× magnification or at least about 40× magnification;
the method further comprises:
evaluating a presence of tumor or cancer cells having anti-PTK7 staining at any intensity above the defined threshold in at least a portion of the tissue sample at the low magnification; and
determining a 1+ or greater positive staining intensity above the defined threshold of the total number of staining viable tumor or cancer cells in the tissue sample at the medium magnification;
the method further comprises determining a 1+ or greater positive staining intensity above the defined threshold of the total number of staining viable tumor or cancer cells in the tissue sample at the high magnification;
the method further comprises the low magnification: (a) evaluating areas of heavy stromal and necrosis anti-PTK7 staining in the tissue sample. (b) determining a distribution of viable tumor cells, cancer cells. and non-neoplastic tissue in the stained tissue sample; or (c) identifying a presence of viable tumor or cancer cells having a 2+ or greater staining intensity;
the method further comprises at the medium or high magnification: (a) distinguishing between cellular membrane positive anti-PTK7 staining and cytoplasmic anti-PTK7 positive staining in the total number of viable tumor or cancer cells in the tissue sample: (b) distinguishing between 1+, 2+, or 3+ positive staining intensities in the total number of viable tumor or cancer cells in the tissue sample; or (c) distinguishing between 0 staining and 1+ positive cellular staining intensities in the tissue sample;
the method further comprises at the high magnification: (a) confirming the cellular membrane versus cytoplasmic positive anti-PTK7 staining for the total number of viable tumor or cancer cells in the tissue sample having a 2+ or greater staining intensity and a heavy level of cytoplasmic staining; or (b) confirming the cellular membrane versus cytoplasmic staining for the total number of viable tumor or cancer cells in the tissue sample having a 1+ or greater staining intensity;
the method further comprises: (a) at the low magnification, confirming a distribution of 0, 1+, 2+, and 3+ positive cellular staining intensities in the tissue sample: optionally, also adjusting the Tumor Proportion Score based on the confirmation;
the tissue sample comprises a tissue section, and it is determined if the tissue section is or is not adequate for the determining and scoring of the amount of expression of protein PTK7, and the tissue section is considered adequate for evaluation if about 100 or more viable invasive tumor or cancer cells are present;
an invasive tumor or cancer cell is counted as positively stained with anti-PTK7 if the invasive tumor or cancer cell displays at an anti-PTK7 cellular membrane staining at any intensity of 1+ or higher;
a section or portion of the tissue sample is prepared on a slide or equivalent, and the section or portion of the tissue sample is stained on the slide;
the antibody which specifically binds to PTK7 comprises a monoclonal mouse anti-PTK7 antibody; or, the anti-PTK7 antibody comprises monoclonal mouse anti-PTK7 clone 6.60.1; or, the anti-PTK7 antibody comprises a substantially isolated or substantially purified monoclonal mouse anti-PTK7 clone 6.60.1;
there is an anti-PTK7 staining at any intensity 1+ and higher, when: (a) the staining signal is unequivocally brown, or (b) the staining corresponds to a cell membrane;
the method further comprises excluding from the calculation of the Tumor Proportion Score (TPS): tumor cells or cancer cells with only cytoplasmic or nuclear staining; non-invasive neoplasia or carcinoma in situ cells, non-viable or necrotic tumor or cancer cells, apoptotic nuclei or nuclear debris, tumor or cancer cells in poorly preserved tissue areas, benign epithelial cells, non-neoplastic cells and/or lymphocytes with nuclear staining, apoptotic cells, necrotic cells, cells not exhibiting an intended color, lymphocytes, and stromal cells:
the method further comprises (i) if the Tumor Proportion Score (TPS) is less than (<) 75%, then the tissue sample is determined to have diagnostic negative PTK7 expression; and (ii) if the Tumor Proportion Score (TPS) is greater than or equal to (>) 75%, then the tissue sample is determined to have diagnostic positive PTK7 expression;
the tissue sample comprises a formalin-fixed, paraffin-embedded (FFPE) specimen; or, a section of the tissue sample is prepared by a protocol comprising fixation in about 10% neutral buffered formalin for between about 6 to 72 hours;
the tumor or cancer is a non-small cell lung cancer (NSCLC), a breast cancer or breast carcinoma, a head and neck cancer, a colorectal cancer, a bladder cancer, a lung cancer, a gastrointestinal stromal tumor (GIST), a prostate cancer, a cervical cancer, an ovarian cancer, or a renal cell carcinoma; and/or
the tissue sample is a biopsy sample, or is or is derived from a needle biopsy sample, a fine-needle aspirate, a cytology specimen, or a bone decalcification.

In alternative embodiments, provided are methods for diagnosing or selecting an individual eligible for treatment with a cancer or tumor therapeutic, comprising determining and scoring the amount of nuclear protein PTK7 (also known as colon carcinoma kinase 4 (CCK4)) in a tissue sample from an individual using an IHC method as provided herein, wherein if the tissue sample is determined or scored to have a high or a diagnostic positive PTK7 expression. the individual is eligible for treatment with a cancer therapeutic to which the individual is likely to respond favorably.

In alternative embodiments of methods herein, the tumor or cancer is a non-small cell lung cancer (NSCLC). a breast cancer or breast carcinoma, a head and neck cancer, a colorectal cancer, a bladder cancer, a lung cancer, a gastrointestinal stromal tumor (GIST), a prostate cancer, a cervical cancer, or a renal cell carcinoma.

In alternative embodiments, provided are methods for diagnosing or selecting an individual eligible for treatment with a cancer or tumor therapeutic, comprising: (a) determining if cells in a tissue sample from the individual in need thereof have a low or a high, or a diagnostic negative or a diagnostic positive. PTK7 expression score (%), as determined by a protocol comprising use of an immunohistochemistry (IHC) method as set forth according to embodiments herein; and, (b) diagnosing or selecting the individual as eligible for treatment with the tumor or a cancer therapeutic if the tissue sample is found to have a high or a diagnostic positive PTK7 expression score (TPS).

In alternative embodiments of methods for diagnosing or selecting an individual eligible for treatment with a cancer or a tumor therapeutic:
the cancer or tumor therapeutic is a non-small cell lung cancer (NSCLC) therapeutic or a lung cancer therapeutic:
the cancer or tumor therapeutic is a pharmaceutical formulation comprising an antibody drug conjugate, a radionuclide, a gallium-68 radionuclide, or a fluorine-18 radionuclide;
the individual is a patient with a lung cancer or a non-small cell lung cancer;
the method comprises determining the number of PTK7 positive viable tumor or cancer cells comprises determining the number of viable tumor or cancer cells having PTK7 staining in the cellular membranes;
the method comprises determining the number of PTK7 positive viable tumor or cancer cells comprises determining the number of viable tumor or cancer cells having a higher intensity cellular membrane PTK7 staining than cytoplasmic PTK7 staining;
the method further comprises determining that the individual is likely to respond favorably to treatment with a cancer therapeutic if the Tumor Proportion Score (TPS) is above a threshold value, and optionally the threshold value is about 50TPS or above, about 75% or above, or about any number between 50% and 90%; and/or
the method further comprises selecting the individual for administration of the cancer therapeutic if the Tumor Proportion Score (TPS) is above the threshold value.

In alternative embodiments, provided are kits comprising an antibody which specifically binds to PTK7, and scoring guidelines comprising or as used in a method as provided herein, for example as used in a method for treating or ameliorating a cancer or a tumor in an individual in need thereof, as provided herein.

In alternative embodiments, provided are kits comprising scoring guidelines comprising or as used in a method as provided herein, for example as used in a method for diagnosing or selecting an individual eligible for treatment with a cancer or tumor therapeutic. as provided herein.

In alternative embodiments of kits as provided herein, the kits further comprise images indicating a plurality of PTK7 staining levels.

In alternative embodiments, provided are methods for assessing the extent of PTK7 expression comprising: contacting a sample or a portion thereof comprising cancer or tumor cells from an individual with an antibody or portion thereof which specifically binds to PTK7; and, determining a Tumor Proportion Score (TPS) by dividing the number of PTK7 staining viable tumor or cancer cells in the sample or portion thereof specifically bound by the antibody with the total number of staining and non-staining viable cancer or tumor cells and multiplying the result by 100, thereby obtaining the Tumor Proportion Score (TPS).

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1 illustrates images of intensity examples from 0-3+ from non-small cell lung cancer (NSCLC), tumor tissue stained with PTK7 demonstrating membrane staining, as described in further detail in Example 1. below.

FIG. 2 provides example images of low, moderate, and high examples at each staining intensity. These images are meant as a guideline and are not intended to represent the full range of staining variation within each intensity category (0-3+), as described in further detail in Example 1. below.

FIG. 3 illustrates a T&T (Training and Testing) Inter-Observer Variability plot: the x-axis demonstrates the 30 tissue blocks that were scored by four observers, or pathologists, as described in further detail in Example 1, below.

FIG. 4A illustrates a visual summary of the various FOVs used in IHC examination in this exemplary protocol, and FIG. 4B illustrates exemplary FOVs as superimposed over the image of a tissue section, as described in further detail in Example 1. below.

Like reference symbols in the various drawings indicate like elements.

### DETAILED DESCRIPTION

In alternative embodiments, provided are immunohistochemistry (IHC) methods and kits for determining and scoring reproducibly the extent of expression of the protein tyrosine-protein kinase-like 7 (PTK7). also known as: colon carcinoma kinase 4 (CCK4): HER2 or receptor tyrosine-protein kinase erbB-2, or cluster of differentiation 340 (CD340); programmed death-ligand 1 (PD-L1), or cluster of differentiation 274 (CD274): B7 homolog 1 (B7-H1); and, Ki-67 or MK167 (Marker of Proliferation Ki-67), in a tissue sample. In alternative embodiments, scoring methods are provided to assess PTK7 expression in tumors or cancers. Certain aspects include use of scoring methods as provided herein to assess PTK7 expression and determine if a high or a diagnostically positive Tumor Proportion Score is present.

Alternative embodiments provide methods and kits for diagnosing or selecting an individual eligible for treatment with a cancer or tumor therapeutic. In certain embodiments, if a tissue sample is determined or scored to have a high or diagnostically positive Tumor Proportion Score, the patient is diagnosed or selected for treatment with a cancer therapeutic to which the patient is likely to respond favorably.

In alternative embodiments, provided are methods and kits for diagnosing, treating or ameliorating or assessing the risk of recurrence for a cancer or a tumor using an IHC method as provided herein.

PTK7 has been found to be expressed in and to contribute to the progression of various cancers and tumors. Such cancers include malignancies with a poor prognosis, including lung cancers and non-small cell lung cancers (NSCLC). PTK7 overexpression has been found to be a promising candidate biomarker to predict the occurrence and prognosis of several types of cancers, not only of lung cancers and NSCLC, but also stage I-IV hepatocellular carcinoma (HCC), invasive breast cancers, cervical cancers, colorectal cancers, and thyroid cancers. PTK7 expression patterns in cancers and tumors versus normal tissues make it an attractive target for development of diagnostic assays.

Currently available CDx scoring guidelines contain a formula for how to interpret IHC staining in tissue samples. Such a formula is written as an equation, such as with CPS (the number of PTK7 positive cells (tumor cells and immune cells)/the number of total tumor cells. There is no method described as to how to determine the CPS score for the entire slide using the numerator and denominator. Pathologists often ask what method should be followed to get the correct score, and there has been no structured way to respond to that question.

Attempts to get correct IHC scores include the use of IHC staining colors and/or thickness to assess intensity; however, disadvantages of such methods include: that (1) color (i.e. dark brown, brown, light brown) is highly subjective, dependent on the scoring method (i.e. glass vs digital) and only suitable for non-intensity-based scoring algorithms; and, (2) thickness is generally specific to membrane staining and is not applicable to all cancer indications and biomarkers.

There remains a need in the art for not only robust, reliable, reproducible. objective and accurate PTK7 scoring methods, but also for scoring methods having greater simplicity and efficiency. Embodiments of immunohistochemistry (IHC) methods herein can provide a considerable advantage for reliable and efficient diagnostic assessments of the extent of expression of PTK7 in tissue samples.

### Embodiments of Immunohistochemistry (IHC) Methods

Embodiments herein are directed to immunohistochemistry methods for determining and scoring the extent of expression of protein PTK7 in a tissue sample. In various embodiments, the method includes: staining a tissue sample with an antibody which specifically binds to PTK7; determining a total number of viable invasive tumor or cancer cells having anti-PTK7 staining, determining a total number of staining and non-staining viable invasive tumor or cancer cells in at least a portion of the tissue sample, wherein an invasive tumor or cancer cell is counted as positively stained with anti-PTK7 if the invasive tumor or cancer cell displays at an anti-PTK7 cellular staining at any intensity above a defined threshold; and determining a Tumor Proportion Score (TPS), wherein the Tumor Proportion Score (TPS) is the number of PTK7 staining viable invasive tumor or cancer cells found in the tissue sample divided by the total number of staining and non-staining viable invasive tumor or cancer cells, multiplied by 100. Such embodiments can provide not only robust, reliable, reproducible, and accurate PTK7 scoring methods, but also scoring methods having greater objectivity, simplicity, and efficiency. Such embodiments can provide a considerable advantage for reliable and efficient diagnostic assessments.

In certain embodiments, the tumor or cancer is a non-small cell lung cancer (NSCLC), a breast cancer or breast carcinoma, a head and neck cancer, a colorectal cancer, a bladder cancer, a lung cancer, a gastrointestinal stromal tumor (GIST), a prostate cancer, a cervical cancer, an ovarian cancer, or a renal cell carcinoma.

Various embodiments provide systematic microscopic methods for arriving at a correct biomarker score using field of views and changing magnifications. Magnification based guidance is an objective approach to assessing IHC intensity. The standardization of magnification across microscopes allows the observer and/or pathologist to follow a specific and unique sequence of steps to ensure they are accurately evaluating staining intensity from 0-3. Such embodiments provide methods for microscopic scoring that can have the advantage of being highly reproducible.

In certain embodiments, the defined threshold includes: a 1+ or greater positive staining intensity evaluated at a low magnification; a 2+ or greater positive staining intensity evaluated at a medium magnification; or a 3+ positive staining intensity evaluated at a high magnification. In some embodiments, the low magnification is at least about 4× magnification; the medium magnification is at least about 10× magnification; and the high magnification is at least about 20× magnification or at least about 40× magnification.

In certain embodiments, the positive staining is determined using a bright-field light microscope, a microscope objective, a computer monitor and imaging software. or a combination thereof. In some aspects. the imaging software comprises whole slide imaging software.

In certain embodiments, the method further includes: evaluating a presence of tumor or cancer cells having anti-PTK7 staining at any intensity above the defined threshold in at least a portion of the tissue sample at the low magnification; and determining a 1+ or greater positive staining intensity above the defined threshold of the total number of staining viable tumor or cancer cells in the tissue sample at the medium magnification. In some embodiments, the method includes determining a 2+ or greater positive staining intensity at the medium magnification. In certain embodiments, the method further includes: determining a 1+ or greater positive staining intensity above the defined threshold of the total number of staining viable tumor or cancer cells in the tissue sample at the high magnification. In some embodiments, the method includes determining a 2+ or greater positive staining intensity at the high magnification.

In certain embodiments, the method further includes: at the low magnification, evaluating areas of heavy stromal and necrosis anti-PTK7 staining in the tissue sample, determining a distribution of viable tumor cells, cancer cells, and non-neoplastic tissue in the stained tissue sample; or identifying a presence of viable tumor or cancer cells having a 2+ or greater staining intensity.

Certain embodiments further include: at the medium or high magnification. distinguishing between cellular membrane positive anti-PTK7 staining and cytoplasmic anti-PTK7 positive staining in the total number of viable tumor or cancer cells in the tissue sample. Such embodiments can provide advantages of increasing the accuracy and reproducibility of PTK7 scoring methods by distinguishing between cells having different structural staining patterns, or excluding staining cells having certain structures or characteristics from the Tumor Proportion Score determination.

Other embodiments of methods herein further include distinguishing between 1+, 2+, or 3+ positive staining intensities in the total number of viable tumor or cancer cells in the tissue sample; or distinguishing between 0 staining and 1+ positive cellular staining intensities in the tissue sample. Such embodiments can provide a benefit of versatility in the staining intensity of the defined threshold.

In certain embodiments, the method further includes: at the high magnification, confirming the cellular membrane versus cytoplasmic positive anti-PTK7 staining for the total number of viable tumor or cancer cells in the tissue sample having a 2+ or greater staining intensity and a heavy level of cytoplasmic staining. Certain embodiments further include confirming the cellular membrane versus cytoplasmic staining for the total number of viable tumor or cancer cells in the tissue sample having a 1+ or greater staining intensity. In some embodiments, a heavy level of cytoplasmic staining includes a level of cytoplasmic PTK7 staining that is equivalent to or greater than the level of membrane PTK7 staining.

In certain embodiments, the method further includes: at the low magnification, confirming a distribution of 0, 1+, 2+, and 3+ positive cellular staining intensities in the tissue sample. Certain embodiments optionally include adjusting the Tumor Proportion Score based on the confirmation.

In certain embodiments, an invasive tumor or cancer cell is counted as positively stained with anti-PTK7 if the invasive tumor or cancer cell displays at an anti-PTK7 cellular membrane staining at any intensity of 1+ or higher.

In certain embodiments, an anti-PTK7 staining at any intensity 1+ and higher is determined, when the staining signal is unequivocally brown. In certain embodiments, an anti-PTK7 staining at any intensity 1+ and higher is determined when the staining corresponds to a cell membrane.

In certain embodiments, the method further includes excluding from the calculation of the Tumor Proportion Score (TPS): tumor cells or cancer cells with only cytoplasmic or nuclear staining: non-invasive neoplasia or carcinoma in situ cells, non-viable or necrotic tumor or cancer cells, apoptotic nuclei or nuclear debris, tumor or cancer cells in poorly preserved tissue areas, benign epithelial cells, non-neoplastic cells and/or lymphocytes with nuclear staining. apoptotic cells, necrotic cells, cells not exhibiting an intended color, lymphocytes, and stromal cells. Such embodiments can provide advantages of increasing the accuracy and reproducibility of PTK7 scoring methods by excluding stained cells having certain structures or characteristics from the Tumor Proportion Score determination.

In certain embodiments of methods herein, if the Tumor Proportion Score (TPS) is less than (<) 75%, then the tissue sample is determined to have diagnostic negative PTK7 expression. In certain embodiments, a Tumor Proportion Score less than 70% indicates a diagnostic negative PTK7 expression. In certain embodiments, a Tumor Proportion Score less than 60% indicates a diagnostic negative PTK7 expression. In certain embodiments, if the Tumor Proportion Score (TPS) is greater than or equal to (≥) 75%, then the tissue sample is determined to have diagnostic positive PTK7 expression. In certain embodiments, a Tumor Proportion Score greater than 75% indicates a diagnostic positive PTK7 expression. In certain embodiments, a Tumor Proportion Score greater than 80% indicates a diagnostic positive PTK7 expression. In certain embodiments, a Tumor Proportion Score greater than 85% indicates a diagnostic positive PTK7 expression.

In certain embodiments, the antibody which specifically binds to PTK7 includes a monoclonal mouse anti-PTK7 antibody. In certain embodiments, the anti-PTK7 antibody comprises monoclonal mouse anti-PTK7 clone 6.60.1. In certain embodiments, the anti-PTK7 antibody comprises a substantially isolated or substantially purified monoclonal mouse anti-PTK7 clone 6.60.1.

In certain aspects of methods herein, the tissue sample includes a tissue section; in certain embodiments, it is determined if the tissue section is or is not adequate for the determining and scoring of the amount of expression of protein PTK7. In certain embodiments, the tissue section is considered adequate for evaluation if about 100 or more viable invasive tumor or cancer cells are present. In certain embodiments, the tissue section is considered adequate for evaluation if about 200 or more viable invasive tumor or cancer cells are present. In certain embodiments, the tissue section is considered adequate for evaluation if about 500 or more viable invasive tumor or cancer cells are present.

In various embodiments, a section or portion of the tissue sample is prepared on a slide or equivalent, and the section or portion of the tissue sample is stained on the slide. In certain embodiments, a section or portion of the tissue sample is prepared on a slide, a microscope slide, or equivalent, and the section or portion of the tissue sample is stained on the slide. In certain embodiments, the tissue sample comprises a formalin-fixed, paraffin-embedded (FFPE) specimen. In certain embodiments, the FFPE specimen comprises a cancer specimen stained on an automated IHC platform. In certain embodiments, the section of the tissue sample is prepared by a protocol comprising fixation in about 10% neutral buffered formalin for a time period of about 6 hours to about 72 hours. In certain embodiments, the tissue sample is a biopsy sample, a needle biopsy sample, a sample derived from a needle biopsy sample, a fine-needle aspirate, a cytology specimen, or a bone decalcification.

### Embodiments of Methods for Diagnosinsg a Tumor or a Cancer

Embodiments herein provide methods for diagnosing a tumor or a cancer by determining if a tissue sample is positive for expression of PTK7. In various aspects, the method comprises: determining a PTK7 diagnostic status in a tissue sample by use of an IHC method for determining the extent of cellular membrane expression of PTK7 as provided herein, where the method can comprise determining if a PTK7 staining is at a defined threshold, which can comprise: a 1+ or greater positive staining intensity evaluated at a low magnification; a 2+ or greater positive staining intensity evaluated at a medium magnification; or a 3+ positive staining intensity evaluated at a high magnification. Such embodiments can provide a benefit of versatility in the staining intensity of the defined threshold. In alternative embodiments, if the Tumor Proportion Score (TPS) is less than (<) 75%, then the tissue sample is determined to have diagnostically negative PTK7 expression; and if the Tumor Proportion Score (TPS) is greater than or equal to (>) 75%, then the tissue sample is determined to have diagnostically positive PTK7 expression. Such embodiments can provide a benefit of accuracy in a diagnostic status determination related to PTK7 expression.

In certain embodiments, the tumor or the cancer is a non-small cell lung cancer (NSCLC), a breast cancer or breast carcinoma, ahead and neck cancer, a colorectal cancer, a bladder cancer, a lung cancer, a gastrointestinal stromal tumor (GIST), a prostate cancer, a cervical cancer, or a renal cell carcinoma.

Embodiments herein provide methods for diagnosing or selecting an individual eligible for treatment with a cancer or tumor therapeutic. In various embodiments, such a method includes determining and scoring the amount of nuclear protein PTK7 (also known as colon carcinoma kinase 4 (CCK4)) in a tissue sample from an individual using an IHC method according to embodiments provided herein, wherein if the tissue sample is determined or scored to have a high or a diagnostic positive PTK7 expression, the individual is diagnosed or selected for treatment with a cancer therapeutic to which the individual is likely to respond favorably. In certain embodiments, the tumor or cancer is a non-small cell lung cancer (NSCLC), a breast cancer or breast carcinoma, a head and neck cancer, a colorectal cancer, a bladder cancer, a lung cancer, a gastrointestinal stromal tumor (GIST), a prostate cancer, a cervical cancer, or a renal cell carcinoma.

Embodiments herein provide a method for diagnosing or selecting an individual eligible for treatment with a cancer or tumor therapeutic. In various embodiments, such a method includes: (a) determining if cells in a tissue sample from the individual have a low or a high, or a diagnostic negative or a diagnostic positive, PTK7 expression score (TPS). as determined by a protocol including use of an immunohistochemistry (IHC) method as set forth in various embodiments herein; and (b) diagnosing or selecting the individual as eligible for treatment with the cancer or tumor therapeutic if the tissue sample is found to have a high or a diagnostic positive PTK7 expression score (TPS). In certain embodiments, the cancer or tumor therapeutic is a non-small cell lung cancer (NSCLC) therapeutic or a lung cancer therapeutic. In certain embodiments, the cancer or tumor therapeutic is a pharmaceutical formulation comprising an antibody drug conjugate. a radionuclide, a gallium-68 radionuclide, or a fluorine-18 radionuclide. In certain embodiments, the individual is a patient with a lung cancer or a non-small cell lung cancer.

In certain embodiments, determining the number of PTK7 positive viable tumor or cancer cells includes determining the number of viable tumor or cancer cells having PTK7 staining in the cellular membranes. In certain embodiments, determining the number of PTK7 positive viable tumor or cancer cells includes determining the number of viable tumor or cancer cells having a higher intensity cellular membrane PTK7 staining than cytoplasmic PTK7 staining.

In certain embodiments, such a method further includes determining that the individual is likely to respond favorably to treatment with a cancer therapeutic if the Tumor Proportion Score (TPS) is above a threshold value. In certain embodiments, the threshold value is about 50% or above, about 75% or above, or about any number between 50% and 90%. In some aspects, the method further includes selecting the individual for administration of the cancer therapeutic if the Tumor Proportion Score (TPS) is above the threshold value.

### Embodiments of Methods for Treating Cancers and Tumors

Provided are methods for treating or ameliorating a tumor or a cancer in a patient. comprising determining and scoring the amount of PTK7 in a tissue sample from the patient using a method as provided herein, wherein if the tissue sample is determined or scored to have a high or a diagnostically positive Tumor Proportion Score, the patient is treated with a cancer therapeutic or an anti-cancer therapy to which the patient is likely to respond favorably.

In certain embodiments, the tumor or the cancer is a non-small cell lung cancer (NSCLC), a breast cancer or breast carcinoma, ahead and neck cancer, a colorectal cancer, a bladder cancer, a lung cancer, a gastrointestinal stromal tumor (GIST), a prostate cancer, a cervical cancer, or a renal cell carcinoma.

In certain embodiments, the cancer therapeutic comprises administration to the patient an anti-cancer drug or an anti-cancer therapy. In alternative embodiments, the anti-cancer treatment or therapy comprises: surgery such as cyberknife therapy: chemo-embolization; an ablation technique such as radiofrequency ablation (RFA), cryoablation and/or microwave ablation; and/or, radiation therapy such as stereotactic body radiation therapy.

In certain embodiments, the anti-cancer therapy comprises an antibody drug conjugate, a small molecule therapy, an immunotherapy, a monoclonal antibody therapy, an adoptive cell therapy, a T-cell receptor therapy, or a chimeric antigen receptor (CAR) T-cell therapy. In alternative embodiments, the anti-cancer treatment or therapy comprises: a tyrosine kinase inhibitor (optionally erlotinib (or TARCEVA™), gefitinib (or IRESSA™), afatinib (or GILOTRIF™), or osimertinib (TAGRISSO™)); necitumumab (or PORTRAZZA™), pembrolizumab (or KEYTRUDA™), nivolumab (or OPDIVO™), ipilimumab (YERVOY™), cetuximab (or ERBITUX™), cisplatin (or PLATINOL™) or carboplatin (or PARAPLATIN™).

In alternative embodiments, the anti-cancer treatment or therapy comprises use of an anti-cancer drug that can comprise an antibody that specifically or substantially binds to the cancer or tumor, wherein the antibody is conjugated to a cytotoxic agent, and optionally the cytotoxic agent comprises a radionuclide (optionally Yttrium-90, Iodine-131, Lutetium-177, Radium-223 chloride, strontium-89 chloride or samarium-153 EDTMP), diphtheria toxin, pseudomonas exotoxin A, denileukin diftitox (or ONTAK™), moxetumomab pasudotox (or LUMOXITI™), inotuzumab ozogamicin (or BESPONSA™), calicheamicin or N-acetyl-γ-calicheamicin, emtansine or DM1, maytansine or derivatives thereof, mertansine. ravtansine. soravtansine or mirvetuximab soravtansine, SN-38 (or 7-Ethyl-10-hydroxycamptothecin), or auristatin or monomethyl auristatin E (MMAE).

### Immunohistochemistry

In alternative embodiments, methods as provided herein can use or comprise reagents for detecting or visualizing an antibody-antigen interaction using any products or methods known in the art. for example, an IHC protocol or reagents.

In alternative embodiments, immunohistochemistry methodologies and/or reagents used with methods and products of manufacture or kits as provided herein can include or comprise or comprise use of any IHC protocol. IHC armamentarium, device and/or image or data analysis system, for practicing IHC or IHC reagents known in the art.

In alternative embodiments, the antibodies, antigen binding fragments thereof, or monomeric or dimeric antigen binding proteins as provided herein (including for example synthetic or recombinant forms) used in IHC protocols, or kits, as provided herein are substantially purified or isolated or are in the form of an unpurified or partially purified culture supernatant.

In alternative embodiments, methods as provided herein comprise use of chromogenic immunohistochemistry (CIH), wherein a primary antibody (for example, a recombinant antibody (Ab), or antigen binding fragment thereof, or monomeric or dimeric antigen binding protein, as provided herein) or secondary antibody (for example, where the secondary antibody binds to the primary antibody, or the recombinant antibody (Ab), or antigen binding fragment thereof, or monomeric or dimeric antigen binding protein as provided herein,) is conjugated to an enzyme such as peroxidase, for example, an immunoperoxidase). for example, a horseradish peroxidase (HRP), that can catalyze a color-producing reaction. In alternative embodiments, a chromogenic moiety used in methods as provided herein is or comprises a coumarin; a rhodamine; 2,3,6,7-tetrahydro-11-oxo-1H,5H,11H-[1]benzopyrano[6,7,8-ij]quinolizine-1-0-carboxylic acid: 7-(diethylamino)coumarin-3-carboxylic acid; a coumarin derivative; a rhodamine derivative; a tetramethylrhodamine; a diarylrhodamine derivative; QSY 7; QSY 9; QSY 21; diazo chromophores; DABSYL; tartrazine; triarylmethane compounds; fast red; fast blue; fuchsin; Cascade Blue acetyl; Dapoxylsulfonic acid/carboxylic acid succinimidyl ester: DY-405: Alexa Fluor 405 succinimidyl ester; Cascade Yellow succinimidvl ester; pyridyloxazole succinimidyl ester (PyMPO); Pacific Blue succinimidyl ester; DY-415; 7-hydroxycoumarin-3-carboxylic acid succinimidyl ester; DYQ-425; 6-FAM phosphoramidite; Lucifer Yellow; iodoacetamide: Alexa Fluor 430 succinimidyl ester: Dabcyl succinimidyl ester: NBD chloride/fluoride; QSY 35 succinimidyl ester; DY-485XL; Cy2 succinimidyl ester: DY-490; Oregon Green 488 carboxylic acid succinimidyl ester; Alexa Fluor 488 succinimidyl ester; BODIPY 493/503 C3 succinimidyl ester; DY-480XL; BODIPY FL C3 succinimidyl ester; BODIPY FL C5 succinimidyl ester; BODIPY FL-X succinimidyl ester; DYQ-505: Oregon Green 514 carboxylic acid succinimidyl ester; DY-510XL; DY-481XL; 6-carboxy-4′,5′-dichloro-2′,7′-dimethoxyfluorescein succinimidyl ester (JOE); DY-520XL: DY-521XL; BODIPY R6G C3 succinimidyl ester; erythrosin isothiocyanate; 5-carboxy-2′,4′,5′,7′-tetrabromosulfonefluorescein succinimidyl ester; Alexa Fluor 532 succinimidyl ester; 6-carboxy-2′,4,4′,5′7,7′-hexachlorofluorescein succinimidyl ester (HEX); BODIPY 530/550 C3 succinimidyl ester; DY-530; BODIPY TMR-X succinimidyl ester: DY-555; DYQ-1; DY-556; Cy3 succinimidyl ester: DY-547; DY-549: DY-550: Alexa Fluor 555 succinimidvl ester: Alexa Fluor 546 succinimidvl ester; DY-548; BODIPY 558/568 C3 succinimidyl ester; Rhodamine red-X succinimidyl ester; QSY 7 succinimidyl ester; BODIPY 564/570 C3 succinimidyl ester: BODIPY 576/589 C3 succinimidyl ester; carboxy-X-rhodamine (ROX); succinimidyl ester; Alexa Fluor 568 succinimidvl ester; DY-590; BODIPY 581/591 C3 succinimidyl ester; DY-591; BODIPY TR-X succinimidyl ester; Alexa Fluor 594 succinimidvl ester; DY-594; carboxynaphthofluorescein succinimidyl ester; DY-605; DY-610; Alexa Fluor 610 succinimidyl ester; DY-615; BODIPY 630/650-X succinimidyl ester: erioglaucine: Alexa Fluor 633 succinimidyl ester: Alexa Fluor 635 succinimidyl ester; DY-634; DY-630; DY-631; DY-632; DY-633; DYQ-2; DY-636; BODIPY 650/665-X succinimidyl ester; DY-635; Cy5 succinimidyl ester; Alexa Fluor 647 succinimidyl ester; DY-647; DY-648; DY-650; DY-654; DY-652; DY-649; DY-651; DYQ-660; DYQ-661; Alexa Fluor 660 succinimidyl ester; Cy5.5 succinimidyl ester; DY-677; DY-675; DY-676; DY-678; Alexa Fluor 680 succinimidyl ester; DY-679; DY-680; DY-682; DY-681; DYQ-3; DYQ-700; Alexa Fluor 700 succinimidyl ester; DY-703; DY-701: DY-704; DY-700; DY-730; DY-731; DY-732; DY-734; DY-750; Cy7 succinimidyl ester; DY-749: DYQ-4; Cy7.5 succinimidyl ester: 7-diethylaminocoumarin-3-carboxylic acid: succinimidyl ester: Dabsyl sulfonyl chloride: fluorescein isothiocyanate (FITC) carboxy succinimidyl ester (DY-495); Rhodamine Green carboxylic acid succinimidyl ester (DY-505); eosin isothiocyanate (EITC): 6-carboxy-2′,4,7,7′-tetrachlorofluorescein succinimidyl ester (TET); carboxyrhodamine 6G succinimidyl ester; carboxytetramethylrhodamine succinimidyl ester (TMR, TAMRA) (DY-554); QSY 9 succinimidyl ester; sulforhodamine B sulfonyl chloride (DY-560); Texas Red (sulforhodamine 101); gallocyanine; Fast Green FCF; Malachite Green; or, a QSY 21 succinimidyl ester.

In alternative embodiments, methods as provided herein comprise use of immunofluorescence, where a primary or a secondary antibody is tagged to a fluorophore, such as fluorescein or fluorescein isothiocyanate (FITC), a triarylmethane dye such as rhodamine or rhodamine derivatives (for example, tetramethylrhodamine (TRITC), rhodamine 6G, rhodamine 123, rhodamine B, carboxytetramethylrhodamine (TAMRA), tetramethylrhodamine (TMR), sulforhodamine 101), aminomethylcoumarin acetate (AMCA), ALEXA™ or DYLIGHT™ fluors, or a fluorophore or dye as described in U.S. patent application no. US 2019/0018018 A1. 3,3′-Diaminobenzidine (DAB) also can be used.

In alternative embodiments, methods as provided herein comprise use of a direct method or one-step staining method where a primary antibody (for example, antibodies (Ab), or antigen binding fragments thereof, or monomeric or dimeric antigen binding proteins as provided herein (including for example synthetic or recombinant forms)) is labeled and reacts directly with an antigen, for example, in a tissue sections. While this technique utilizes only one antibody and therefore is simple and rapid. the sensitivity may be lower due to little signal amplification.

In alternative embodiments, methods as provided herein comprise use of a direct method or one-step staining method where a primary antibody (for example, antibodies (Ab), or antigen binding fragments thereof, or monomeric or dimeric antigen binding proteins as provided herein (including for example synthetic or recombinant forms)) is labeled and reacts directly with an antigen, for example, in tissue sections. While this technique utilizes only one antibody and therefore is simple and rapid, the sensitivity may be lower due to little signal amplification.

In alternative embodiments, methods as provided herein comprise use of an indirect method where an unlabeled primary antibody (first layer) binds to a target antigen (for example, PTK7 protein), for example, in a tissue or organ. and a labeled secondary antibody (second layer) then is reacted with the primary antibody. The secondary antibody can be against the isotype, for example, IgG, of the animal species in which the primary antibody is derived. This method can be more sensitive than direct detection strategies because of signal amplification due to the binding of several secondary antibodies to each primary antibody if the secondary antibody is conjugated to a detecting agent such as a fluorescent or enzyme reporter.

In alternative embodiments, further amplification is achieved if the secondary antibody is conjugated to several detecting molecules. for example, biotin molecules, which can recruit complexes of avidin-, streptavidin- or NEUTRAVIDIN™ protein-bound enzyme.

In alternative embodiments, the IHC is performed on tissue sections or tissue biopsies, for example, paraformaldehyde (PFA) fixed tissues or organs, or formalin-fixed paraffin-embedded tissues. In alternative embodiments, a tissue is sectioned or sliced or used whole. Before sectioning, the tissue sample can be embedded in a medium, for example, paraffin wax or cryomedia. Tissue sections can be sectioned or sliced on a variety of instruments, most commonly using a microtome, cryostat, or vibratome. Specimens can be sectioned or sliced at a range of about 3 μm to 5 μm. The sections or slices can be mounted on slides, dehydrated using alcohol washes of increasing concentrations (for example, 50%, 75%, 90%, 95%, 100%), and cleared using a detergent like xylene before being imaged under a microscope.

Depending on the method of fixation and tissue preservation, the sample may require additional steps to make a PTK7 epitope available for antibody binding, including deparaffinization and antigen retrieval. For formalin-fixed paraffin-embedded tissues, antigen-retrieval is often necessary, and can comprise pre-treating the sections with heat or proteases.

In alternative embodiments, the IHC is performed using an ENVISION DUOFLEX DOUBLESTAIN SYSTEM™ (EnVision DuoFLEX Doublestain) System) (Agilent, San Jose, CA), which allows for staining of two or more markers on a single slide. In alternative embodiments, the IHC is performed using an EnVision FLEX HRP Magenta, High pH (Dako Omnis) system, and binding can be visualized by EnVision FLEX HRP Magenta Chromogen. In alternative embodiments, the IHC is performed using EnVision FLEX™ MINI™ kit, High pH, which is a high-sensitivity visualization system intended for use in IHC together with Dako AUTOSTAINER™ instruments; this dual link system detects primary mouse and rabbit antibodies and the reaction is visualized by 3,3-Diaminobenzidine (DAB) chromogen (DAB forms a water-insoluble brown precipitate when oxidized, for example, by a peroxidase).

### Products of Manufacture and Kits

Provided are products of manufacture and kits for practicing methods as provided herein; and optionally, products of manufacture and kits can further comprise instructions for practicing methods as provided herein.

Embodiments of a kit herein include an antibody which specifically binds to PTK7, and scoring guidelines including an immunohistochemistry (IHC) method for determining and scoring the extent of expression of protein tyrosine-protein kinase-like 7 (PTK7) in a tissue sample, as provided herein. Other embodiments of a kit herein include scoring guidelines comprising an IHC method as provided herein. In some embodiments, a kit further includes images indicating a plurality of PTK7 staining levels.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%. 18%, 17%. 16%. 15%. 14%. 13%. 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

The invention will be further described with reference to the examples described herein: however. it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition. Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Exemplary IHC Methods

This example describes exemplary IHC methods as provided herein.

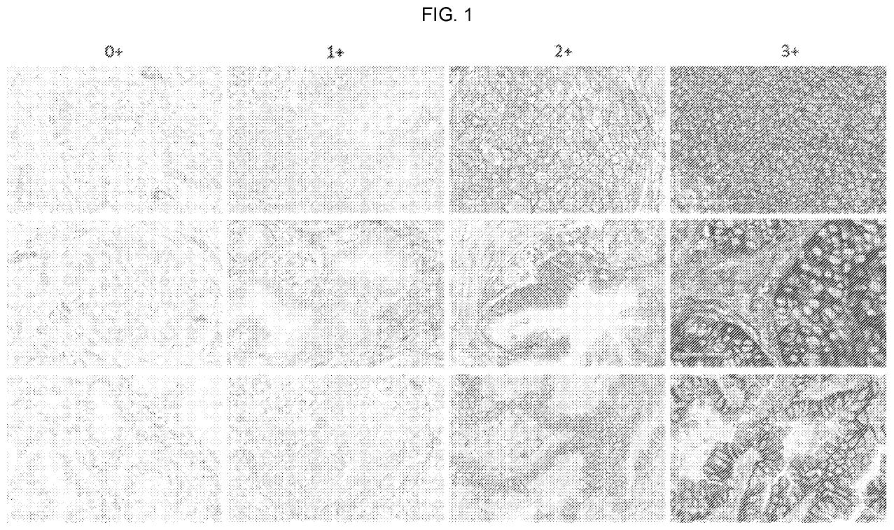

FIG. 1 illustrates images of intensity examples from 0-3+ from non-small cell lung cancer (NSCLC), tumor tissue stained with PTK7 demonstrating membrane staining.

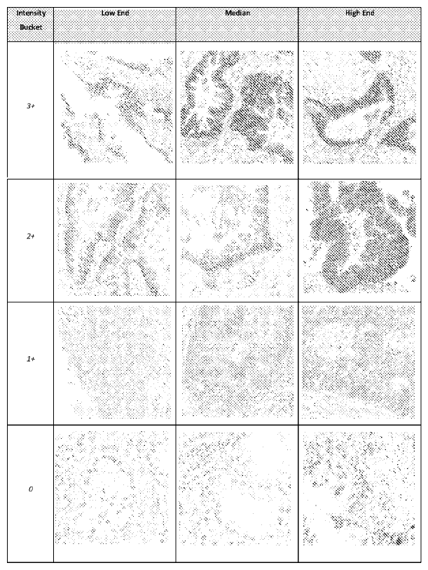

FIG. 2 provides example images of low, moderate, and high examples at each staining intensity. These images are meant as a guideline and are not intended to represent the full range of staining variation within each intensity category (0-3+).

### Membrane Staining Magnification Guidance

The first bullet point of each staining intensity from 1-3 includes a description of color (dark, golden/medium, and light brown). These descriptions are commonly included for non-intensity-based scoring guidelines where any intensity from 1-3 is considered positive staining.

### Stepwise Approach for Assessing Intensity:

TABLE 1

| 1. | Scan specimen at 4X | |
|---|---|---|
| | a. | Determine distribution of tumor and nonneoplastic tissue. |
| | b. | Assess for areas with heavy stromal and necrosis staining. |
| | c. | Observe the relative presence or absence of cells staining at 2+ and 3+ |
| | | within areas of viable tumor |
| 2. | Review tissue at 10X | |
| | a. | Distinguish between 1+, 2+ and 3+ |
| | b. | Confirm membrane versus cytoplasmic staining for all intensities |
| | c. | Record cells staining at 1+, 2+ and 3+ after reviewing all viable tumor |
| 3. | Move to 20X if necessary | |
| | a. | Confirm between 0 and 1+ |
| | b. | Confirm membrane versus cytoplasmic staining for 2+ and 3+ if heavy |
| | | cytoplasmic staining is present |
| | c. | Confirm membrane versus cytoplasmic staining for 1+ |

| | |
|---|---|
| 4. | Move back to 4X |

a. Confirm distribution of 0, 1+, 2+ and 3+ among viable tumor cells

(1+ is more difficult to assess at this magnification)

b. Adjust scores if necessary.

For intensity-based scoring, positive staining is only defined at certain intensities. This positive staining information is used to determine the diagnostic status of a patient sample. Specific magnification guidance included in the second and third bullet points:

TABLE 2

| Staining intensity | Membrane Staining Magnification Guidance |
|---|---|
| Strong (3+) | very dark brown to black color of membrane easily visible at 4X. |
| | 10X objective should be used to distinguish from 2+, and to confirm membrane |
| | localization (versus cytoplasm and stromal) |
| | 20X objective may be necessary to distinguish membrane from cytoplasmic localization |
| | and viable tumor from stromal staining in areas with heavy cytoplasmic staining |
| Moderate (2+) | Golden or medium brown color of membrane staining visible at 4X. |
| | Membrane staining may appear like 3+ at 4X. |
| | 10X objective may be necessary to distinguish from 3+, and to confirm membrane |
| | localization (versus cytoplasm and stromal) |
| | 20X objective may be necessary to distinguish membrane from cytoplasmic localization |
| | and viable tumor from stromal staining in areas with heavy cytoplasmic staining |
| Weak (1+) | Light brown color of membrane visible at 10X-20X but difficult at 4X. |
| | 10X objective should be used to distinguish 1+ from 0 and 2+ and to confirm membrane |
| | versus cytoplasm localization |
| | 20X objective may be necessary to distinguish 1+ from 0 and to confirm membrane |
| | versus cytoplasm localization |
| None (0) | No membrane staining observed at any magnification. |

### Inter-Observer Variability

During training, pathologists are given Table 1 and Table 2, above, in the form of a pathology scoring manual. After training, pathologists are tested for their accuracy in scoring. The pathologists are provided with a form that includes the following table (Table 3) to complete for each specimen:

TABLE 3
0 = no staining, 1+ = weak staining, 2+ = moderate staining,
3+ = strong staining. Total (0-3+) must equal 100%

| | | | | | Total |
|---|---|---|---|---|---|
| Intensity | 0 | 1+ | 2+ | 3+ | (Must equal 100%) |

% of tumor cells with
membrane staining at each
PTK7 intensity

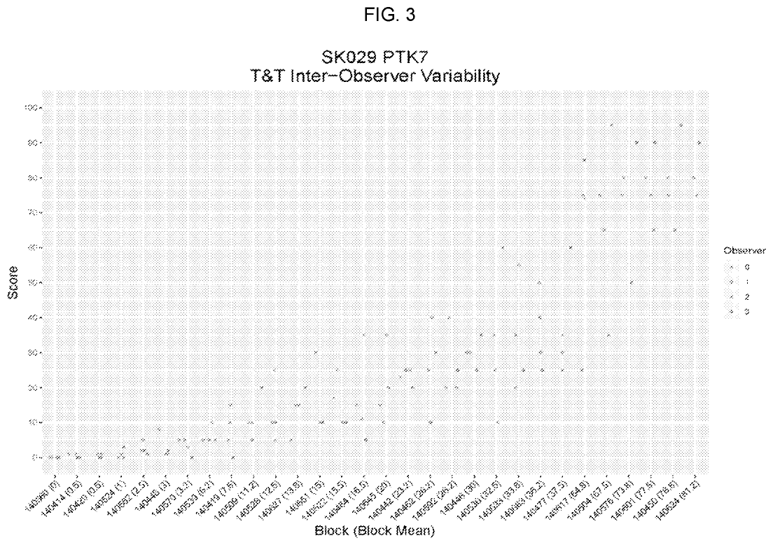

FIG. 3 illustrates a T&T (Training and Testing) Inter-Observer Variability plot: the x-axis demonstrates the 30 tissue blocks that were scored by four observers, or pathologists. The y-axis demonstrates the score for percent of tumor cells greater than or equal to 2+. For example, block 140446 received a score of 25, two scores of 30, and 1 score of 35. This demonstrates that four different pathologists, evaluating the blinded slide with no reference score, were all within 10% for that particular specimen.

### Exemplary Protocol

Examine 40× field of view (FOV), and determine score following guidelines as provided herein;
Change objective to 20×. Examine FOV. Determine score by evaluating the outer FOV especially. Weak or non-staining areas cellular areas may be evaluated at higher magnification for confirmation. Ignore non-cellular areas. Re-evaluate/re-adjust score.
Change objective to 10×. Repeat the above.
Change objective to 4×. Repeat the above.
The result is a score at 4×, which can be repeated for other 4× FOV's if the tissue is large enough. The 4× FOV can be summed and divided by the number of fields for an average.

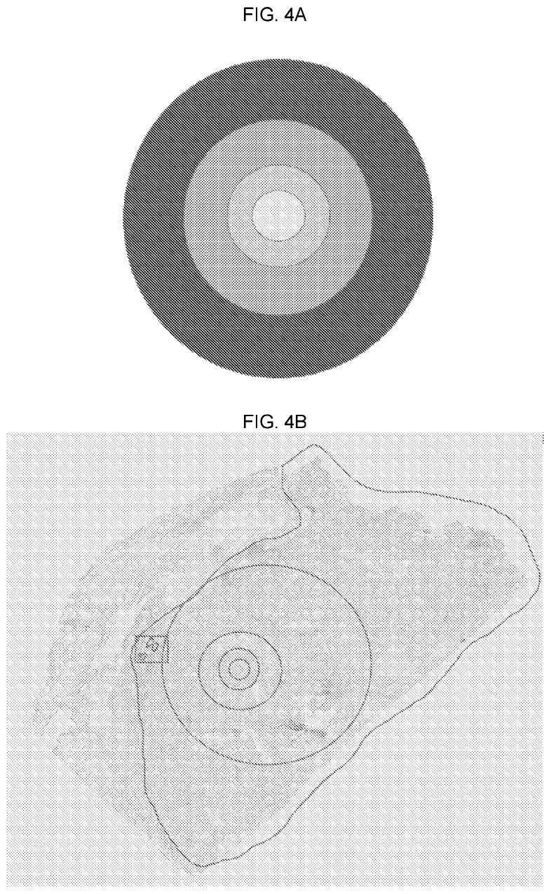

FIG. 4A illustrates a visual summary of the various FOVs used in IHC examination in this exemplary protocol. and FIG. 4B illustrates exemplary FOVs as superimposed over the image of a tissue section.

### EXEMPLARY EMBODIMENTS

Embodiment 1. A method for treating or ameliorating a cancer or a tumor in an individual in need thereof, comprising:
(a) determining if cells in a tissue sample from the individual in need thereof have a low or a high, or a diagnostic negative or a diagnostic positive, PTK7 expression score (TPS), as determined by a protocol comprising use of an immunohistochemistry (IHC) method as set forth in any of claims 1 to 24; and
(b) administering a tumor or a cancer therapeutic to the individual if the tissue sample is found to have a high or a diagnostic positive PTK7 expression score (TPS).

Embodiment 2. The method of Embodiment 1, wherein the cancer or tumor is a non-small cell lung cancer (NSCLC) or a lung cancer.

Embodiment 3. The method of Embodiments 1 or 2, wherein the treating or amelioration of the cancer or tumor comprises administering to the individual in need thereof a pharmaceutical formulation comprising an antibody drug conjugate. a radionuclide, a gallium-68 radionuclide, or a fluorine-18 radionuclide.

Embodiment 4. The method of any of Embodiments 1 to 3, wherein the individual in need thereof is a patient with a lung cancer or a non-small cell lung cancer.

Embodiment 5. The method of any of Embodiments 1 to 4, wherein determining the number of PTK7 positive viable tumor or cancer cells comprises determining the number of viable tumor or cancer cells having PTK7 staining in the cellular membranes.

Embodiment 6. The method of any of Embodiments 1 to 5, wherein determining the number of PTK7 positive viable tumor or cancer cells comprises determining the number of viable tumor or cancer cells having a higher intensity cellular membrane PTK7 staining than cytoplasmic PTK7 staining.

Embodiment 7. The method of any of Embodiments 1 to 6, further comprising determining that a subject is likely to respond favorably to treatment with a cancer therapeutic if the Tumor Proportion Score (TPS) is above a threshold value.

Embodiment 8. The method of Embodiment 7, wherein, the threshold value is about 50% or above, about 75% or above, or about any number between 50% and 90%.

Embodiment 9. The method of any of Embodiments 1 to 8, further comprising administering the cancer therapeutic if the Tumor Proportion Score (TPS) is above the threshold value.

Embodiment 10. A method for treating or ameliorating a cancer or a tumor in a patient, comprising determining and scoring the amount of nuclear protein PTK7 (also known as colon carcinoma kinase 4 (CCK4)) in a tissue sample from a patient in need thereof using an IHC method of any of Embodiments 1-9. wherein if the tissue sample is determined or scored to have a high or a diagnostic positive PTK7 expression. the patient is treated with a cancer therapeutic to which the patient is likely to respond favorably.

Embodiment 11. The method of Embodiment 10, wherein the tumor or cancer is a non-small cell lung cancer (NSCLC), a breast cancer or breast carcinoma, a head and neck cancer, a colorectal cancer, a bladder cancer, a lung cancer, a gastrointestinal stromal tumor (GIST), a prostate cancer. a cervical cancer, or a renal cell carcinoma.

A number of embodiments of the invention have been described. Nevertheless, it can be understood that various modifications may be made without departing from the spirit and scope of the invention. Accordingly, other embodiments are within the scope of the following claims.

1. An immunohistochemistry (JHC) method for determining and scoring the extent of expression of protein tyrosine-protein kinase-like 7 (PTK7) in a tissue sample, the method comprising:
(a) staining a tissue sample with an antibody which specifically binds to PTK7; and
(b) determining a total number of viable invasive tumor or cancer cells having anti-PTK7 staining, and determining a total number of staining and non-staining viable invasive tumor or cancer cells in at least a portion of the tissue sample,
wherein an invasive tumor or cancer cell is counted as positively stained with anti-PTK7 if the invasive tumor or cancer cell displays at an anti-PTK7 cellular staining at any intensity above a defined threshold, and
(c) determining a Tumor Proportion Score (TPS),
wherein the Tumor Proportion Score (TPS) is the number of PTK7 staining viable invasive tumor or cancer cells found in the tissue sample divided by the total number of staining and non-staining viable invasive tumor or cancer cells, multiplied by 100.

2. The method of claim 1, wherein the defined threshold comprises:
a 1+ or greater positive staining intensity evaluated at a low magnification;
a 2+ or greater positive staining intensity evaluated at a medium magnification; or
a 3+ positive staining intensity evaluated at a high magnification.

3. The method of claim 2, wherein the low magnification is at least about 4× magnification; the medium magnification is at least about 10× magnification; and the high magnification is at least about 20× magnification or at least about 40× magnification.

4. The method of claim 2, further comprising:
evaluating a presence of tumor or cancer cells having anti-PTK7 staining at any intensity above the defined threshold in at least a portion of the tissue sample at the low magnification; and
determining a 1+ or greater positive staining intensity above the defined threshold of the total number of staining viable tumor or cancer cells in the tissue sample at the medium magnification.

5. The method of claim 4, further comprising:
determining a 1+ or greater positive staining intensity above the defined threshold of the total number of staining viable tumor or cancer cells in the tissue sample at the high magnification.

6. The method of claim 2, further comprising:
at the low magnification,
(a) evaluating areas of heavy stromal and necrosis anti-PTK7 staining in the tissue sample,
(b) determining a distribution of viable tumor cells, cancer cells, and non-neoplastic tissue in the stained tissue sample; or
(c) identifying a presence of viable tumor or cancer cells having a 2+ or greater staining intensity.

7. The method of claim 2, further comprising:
at the medium or high magnification,
(a) distinguishing between cellular membrane positive anti-PTK7 staining and cytoplasmic anti-PTK7 positive staining in the total number of viable tumor or cancer cells in the tissue sample;
(b) distinguishing between 1+, 2+, or 3+ positive staining intensities in the total number of viable tumor or cancer cells in the tissue sample; or
(c) distinguishing between 0 staining and 1+ positive cellular staining intensities in the tissue sample.

8. The method of claim 7, further comprising:
(a) at the high magnification,
(i) confirming the cellular membrane versus cytoplasmic positive anti-PTK7 staining for the total number of viable tumor or cancer cells in the tissue sample having a 2+ or greater staining intensity and a heavy level of cytoplasmic staining; or
(ii) confirming the cellular membrane versus cytoplasmic staining for the total number of viable tumor or cancer cells in the tissue sample having a 1+ or greater staining intensity; or
(b) at the low magnification, confirming a distribution of 0, 1+, 2+, and 3+ positive cellular staining intensities in the tissue sample; and optionally, adjusting the Tumor Proportion Score based on the confirmation.

9. The method of claim 1, wherein
(a) the tissue sample comprises a tissue section, and it is determined if the tissue section is or is not adequate for the determining and scoring of the amount of expression of protein PTK7, and the tissue section is considered adequate for evaluation if about 100 or more viable invasive tumor or cancer cells are present;
(b) an invasive tumor or cancer cell is counted as positively stained with anti-PTK7 if the invasive tumor or cancer cell displays at an anti-PTK7 cellular membrane staining at any intensity of 1+ or higher;
(c) a section or portion of the tissue sample is prepared on a slide or equivalent, and the section or portion of the tissue sample is stained on the slide;
(d) the antibody which specifically binds to PTK7 comprises a monoclonal mouse anti-PTK7 antibody, and optionally the anti-PTK7 antibody comprises monoclonal mouse anti-PTK7 clone 6.60.1, and optionally the anti-PTK7 antibody comprises a substantially isolated or substantially purified monoclonal mouse anti-PTK7 clone 6.60.1;
(e) there is an anti-PTK7 staining at any intensity 1+ and higher, when:
(i) the staining signal is unequivocally brown, or
(ii) the staining corresponds to a cell membrane.
(f) the method further comprises excluding from the calculation of the Tumor Proportion Score (TPS): tumor cells or cancer cells with only cytoplasmic or nuclear staining; non-invasive neoplasia or carcinoma in situ cells, non-viable or necrotic tumor or cancer cells, apoptotic nuclei or nuclear debris, tumor or cancer cells in poorly preserved tissue areas, benign epithelial cells, non-neoplastic cells and/or lymphocytes with nuclear staining, apoptotic cells, necrotic cells, cells not exhibiting an intended color, lymphocytes, and stromal cells;
(g) the method further comprises:
(i) if the Tumor Proportion Score (TPS) is less than (<) 75%, then the tissue sample is determined to have diagnostic negative PTK7 expression; and
(ii) if the Tumor Proportion Score (TPS) is greater than or equal to (>) 75%, then the tissue sample is determined to have diagnostic positive PTK7 expression.
(h) the tissue sample comprises a formalin-fixed, paraffin-embedded (FFPE) specimen; and optionally a section of the tissue sample is prepared by a protocol comprising fixation in about 10% neutral buffered formalin for between about 6 to 72 hours;
(i) the tumor or cancer is a non-small cell lung cancer (NSCLC), a breast cancer or breast carcinoma, a head and neck cancer, a colorectal cancer, a bladder cancer, a lung cancer, a gastrointestinal stromal tumor (GIST), a prostate cancer, a cervical cancer, an ovarian cancer, or a renal cell carcinoma; and/or
(j) the tissue sample is a biopsy sample, a needle biopsy sample, a sample derived from a needle biopsy sample, a fine-needle aspirate, a cytology specimen, or a bone decalcification.

10. A method for diagnosing or selecting an individual eligible for treatment with a cancer or tumor therapeutic, comprising determining and scoring the amount of nuclear protein PTK7 (also known as colon carcinoma kinase 4 (CCK4)) in a tissue sample from an individual using an IHC method of claim 1, wherein if the tissue sample is determined or scored to have a high or a diagnostic positive PTK7 expression, the individual is diagnosed or selected for treatment with a cancer therapeutic to which the individual is likely to respond favorably.

11. The method of claim 10, wherein the tumor or cancer is a non-small cell lung cancer (NSCLC), a breast cancer or breast carcinoma, a head and neck cancer, a colorectal cancer, a bladder cancer, a lung cancer, a gastrointestinal stromal tumor (GIST), a prostate cancer, a cervical cancer, or a renal cell carcinoma.

12. A method for diagnosing or selecting an individual eligible for treatment with a cancer or tumor therapeutic, comprising:
(a) determining if cells in a tissue sample from the individual in need thereof have a low or a high, or a diagnostic negative or a diagnostic positive, PTK7 expression score (TPS), as determined by a protocol comprising use of an immunohistochemistry (IHC) method as set forth in claim 1; and
(b) diagnosing or selecting the individual as eligible for treatment with the cancer or tumor therapeutic if the tissue sample is found to have a high or a diagnostic positive PTK7 expression score (TPS).

13. The method of claim 12, wherein the cancer or tumor therapeutic is a non-small cell lung cancer (NSCLC) therapeutic or a lung cancer therapeutic; or, the cancer or tumor therapeutic is a pharmaceutical formulation comprising an antibody drug conjugate, a radionuclide, a gallium-68 radionuclide, or a fluorine-18 radionuclide.

14. The method of claim 12, wherein the individual is a patient with a lung cancer or a non-small cell lung cancer.

15. The method of claim 12, wherein determining the number of PTK7 positive viable tumor or cancer cells comprises determining the number of viable tumor or cancer cells having PTK7 staining in the cellular membranes; or, determining the number of PTK7 positive viable tumor or cancer cells comprises determining the number of viable tumor or cancer cells having a higher intensity cellular membrane PTK7 staining than cytoplasmic PTK7 staining.

16. The method of claim 12, further comprising:
(a) determining that the individual is likely to respond favorably to treatment with a cancer therapeutic if the Tumor Proportion Score (TPS) is above a threshold value, wherein optionally the threshold value is about 50% or above, about 75% or above, or about any number between 50% and 90%; or
(b) selecting the individual for administration of the cancer therapeutic if the Tumor Proportion Score (TPS) is above the threshold value.

17. A kit comprising an antibody which specifically binds to PTK7, and scoring guidelines comprising a method of claim 1, and optionally the kit further comprises images indicating a plurality of PTK7 staining levels.

18. A kit comprising scoring guidelines comprising a method of claim 1, and optionally the kit further comprises images indicating a plurality of PTK7 staining levels.

19. A method for assessing the extent of PTK7 expression comprising: contacting a sample or a portion thereof comprising cancer or tumor cells from an individual with an antibody or portion thereof which specifically binds to PTK7; and, determining a Tumor Proportion Score (TPS) by dividing the number of PTK7 staining viable tumor or cancer cells in the sample or portion thereof specifically bound by the antibody with the total number of staining and non-staining viable cancer or tumor cells and multiplying the result by 100, thereby obtaining the Tumor Proportion Score (TPS).
